(19) United States
(12) Patent Application Publication
(10) Pub. No.: US 2021/0307976 A1
(43) Pub. Date: Oct. 7, 2021

(54) LEAK-PROOF WASTE CONTAINMENT DEVICE

(71) Applicant: Jaime Ornelas, Los Angeles, CA (US)

(72) Inventor: Jaime Ornelas, Los Angeles, CA (US)

(21) Appl. No.: 17/221,085

(22) Filed: Apr. 2, 2021

Related U.S. Application Data
(60) Provisional application No. 63/004,593, filed on Apr. 3, 2020.

(51) Int. Cl. A61F 13/56; A61F 13/49; A61F 13/511

(52) U.S. Cl. CPC A61F 13/5655; A61F 13/511; A61F 13/49011

(57) ABSTRACT

A leak-proof waste containment device including a diaper portion, a waist extension, a pair of leg extensions, a plurality of elastic bands and a top protective liner and an absorbent layer beneath the top protective liner. The top protective liner and the absorbent liner is integrated throughout the diaper portion, the waist extension and the pair of leg extensions. The diaper portion, the waist extension, and the pair of leg extensions collectively form an undergarment.

### CROSS REFERENCE TO RELATED APPLICATIONS

This application claims the benefit of priority of U.S. Provisional Application No. 63/004,593 filed on Apr. 3, 2020, the disclosure of which is incorporated herein by reference in its entirety.

### FIELD OF THE INVENTION

Embodiments described herein generally relate to waste containment devices, and more particularly to leak-proof waist containment devices.

### BACKGROUND OF THE INVENTION

Providing appropriate bodily waste management is a major component of sanitation. Waste containment devices, such as, diapers are commonly used for waste management. However, most diapers simply do not hold waste and leakage is unavoidable. This results in embarrassment and discomfort for adults or adolescents who wear waste containment devices and a lengthy cleanup process for all persons who wear diapers (e.g., toddlers, children, persons with special needs and elderly persons) and/or their caregivers. Hence, an improved waste containment device is desirable that prevents or minimizes waste leakage.

### BRIEF DESCRIPTION OF THE DRAWINGS

The various advantages of the embodiments of the present disclosure will become apparent to one skilled in the art by reading the following specification and appended claims, and by referencing the following drawings, in which:

FIG. 1 shows an exemplary view of a leak-proof waste containment device in a closed position according to an embodiment of the present disclosure.

FIG. 2 shows an exemplary view of the leak-proof waste containment device in an open position according to an embodiment of the present disclosure.

FIG. 3 shows an exemplary view of a leak-proof waste containment device in a closed position according to another embodiment of the present disclosure.

FIG. 4 shows an exemplary view of the leak-proof waste containment device in an open position according to another embodiment of the present disclosure

### SUMMARY OF THE INVENTION

Exemplary embodiments disclosed herein describe a leak-proof waste containment device that is configured to minimize waste leakage and to manage human waste effectively. The device includes extensions that wrap around the wearer's waist and thigh respectively, thereby creating extended coverage for waste containment. The device includes a diaper portion, a waist extension, a pair of leg extensions, a plurality of elastic bands and a top protective liner and an absorbent layer beneath the top protective liner. The top protective liner and the absorbent liner is integrated throughout the diaper portion, the waist extension and the pair of leg extensions. The diaper portion, the waist extension, and the pair of leg extensions collectively form an undergarment.

In some exemplary embodiments, the waist extension includes a pair of fasteners.

In some exemplary embodiments the pair of leg extensions each include a pair of fasteners.

In some exemplary embodiments, the pair of fasteners are configured to secure the device to a wearer's waist.

In some exemplary embodiments, the pair of fasteners are configured to secure the device to each of a wearer's leg.

In some exemplary embodiments, the waist extension includes an elastic band.

In some exemplary embodiments, the pair of leg extensions each include an elastic band.

In some exemplary embodiments, the waist extension elastic band is configured to reinforce securement of the device to a wearer's waist to prevent leakage therefrom.

In some exemplary embodiments, the pair of leg extension elastic bands are configured to reinforce securement of the device to each of a wearer's leg to prevent leakage therefrom.

In some exemplary embodiments, each leg extension extends down a wearer's thigh.

Other exemplary embodiments disclosed herein describe a leak-proof waste containment device that is configured to minimize waste leakage and to manage human waste effectively. The device includes extensions that wrap around the wearer's waist and thigh respectively, thereby creating extended coverage for waste containment. The device includes a pull-up diaper portion and a pair of leg extensions, a plurality of fasteners, a plurality of elastic bands and a top protective liner and an absorbent layer beneath the top protective liner. The top protective liner and the absorbent liner is integrated throughout the pull-up diaper portion and the pair of leg extensions. The pull-up diaper portion and the pair of leg extensions collectively form an undergarment.

### DETAILED DESCRIPTION

The disclosure will be described more fully hereinafter with reference to the accompanying drawings, in which exemplary embodiments of the disclosure are shown. This disclosure may, however, be embodied in many different forms and should not be construed as limited to the exemplary embodiments set forth herein. It will be apparent to persons skilled in the relevant art that various changes in form and detail can be made to various embodiments without departing from the spirit and scope of the present disclosure. Thus, the breadth and scope of the present disclosure should not be limited by any of the above-described exemplary embodiments but should be defined only in accordance with the following claims and their equivalents.

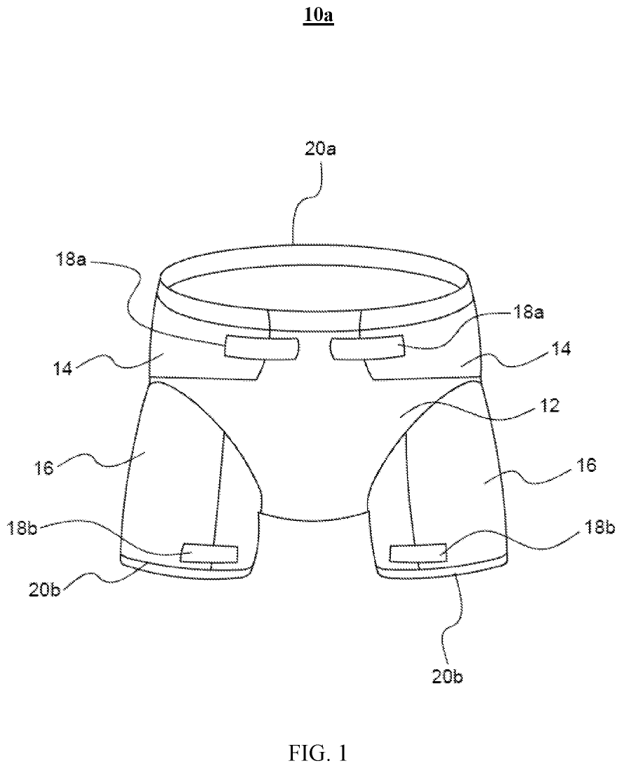

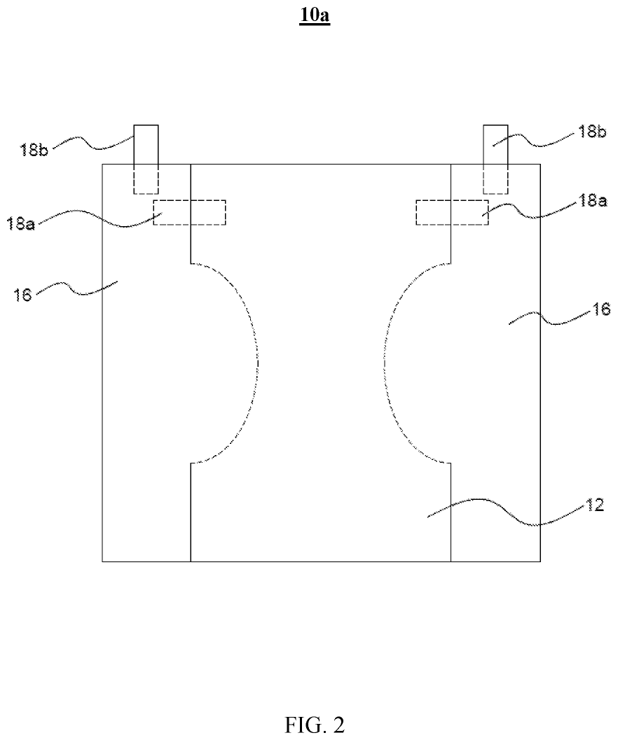

The present disclosure relates to a leak-proof waste containment device (“the device”). As illustrated in FIGS. 1-2, the device 10a includes a diaper portion 12, a waist extension 14, a pair of leg extensions 16, a plurality of fasteners, and a plurality of elastic bands 20. The device in FIG. 1 is shown in a closed configuration. The device in FIG. 2 is shown in an open configuration. When the device is closed, the device is attached to a wearer's body.

The device 10a when worn has an appearance similar to a boxer brief under garment or shorts, where the diaper portion wraps around the wearer in a manner similar to how undergarments are worn, the waist extension extends around the wearer's waist in a high waist manner and the leg extensions extend down the wearer's thighs. The device 10a may be made of any synthetic disposable material(s). The external surface of the device may include a design imprinted thereon. The design may include any suitable images such, as stars, moons, characters, cartoons, logos, etc.

The inside of the device 10a (i.e., the surface in direct contact with the wearer's skin) includes a top protective liner and an absorbent layer beneath the top protective liner. The diaper portion, waist extension and leg extensions each include a top protective liner and absorbent layer beneath the protective liner. The waist extension 14 operates to contain any waste that has leaked from the top region of diaper portion 12. The pair of leg extensions are configured to contain waste (i.e., any waste that has leaked from the bottom of the diaper portion) and thereby prevent any leakage therefrom.

To secure the donning of the device 10a by the wearer, a plurality of resealable fasteners 18 (e.g., fastener tape) are provided on the waist extension 14 and the pair of leg extensions 16. The fasteners 18a provided on the waist extension 14 provide a secure attachment of the device 10a to the wearer at the wearer's waist. A fastener 18a may be located on each end of the waist extension 14 and both may attach to a front region of diaper portion 12. The fasteners 18b provided on the pair of leg extensions 16 each provide a secure attachment of the device 10a to the wearer at each of the wearer's thigh. A fastener 18b may be located on each leg extension and each fastener may close at a center region of the respective thigh. The attachment of the waist extension 14 and the pair of leg extensions to the wearer should be snug so as to prevent any possible leakage. Any suitable resealable fasteners may be used, such as, for example, fastener tape, Velcro strips, etc.

Moreover, the waist extension 14 and the pair of leg extensions 16 may each include an elastic band 20a and 20b respectively. The elastic bands 20 are configured to reinforce the attachment of the device 10 to the wearer and further prevents any leakage from the device 10a.

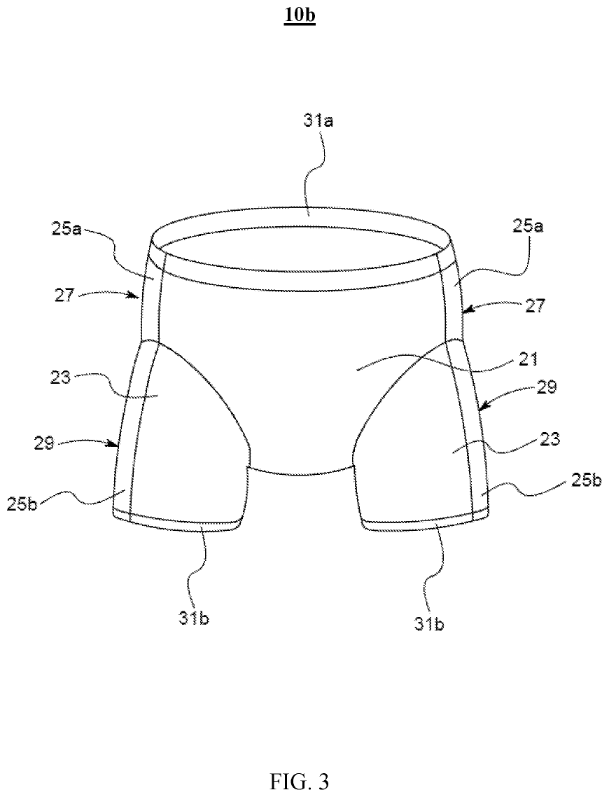

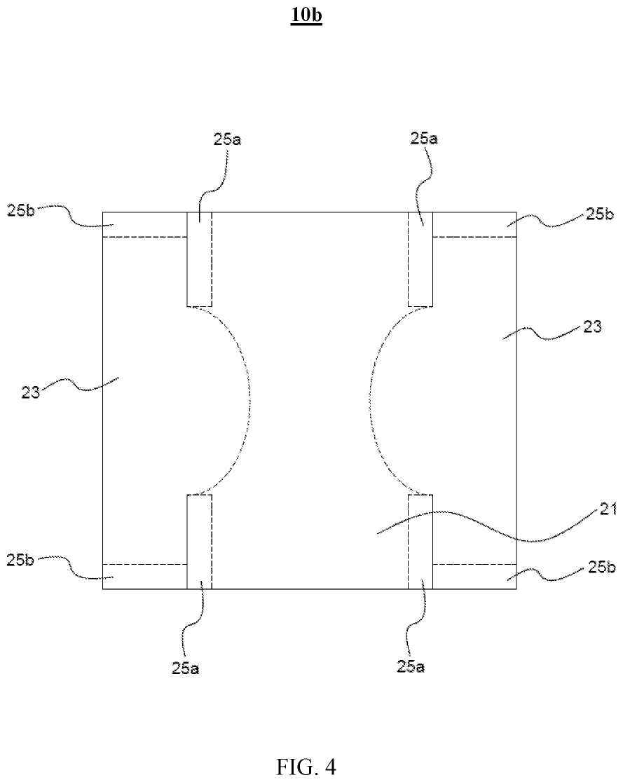

In another embodiment, as illustrated in FIG. 3, the device 10b includes a pull up diaper portion 21, a pair of leg extensions 23 and a plurality of fasteners 25. The device 10b in FIG. 3 is shown in a closed configuration. The device in FIG. 4 is shown in an open configuration. When the device 10b is closed, the device is attached to a wearer's body and has the appearance of a boxer brief under garment or shorts.

The inside of the device 10b includes a top protective liner and an absorbent layer beneath the top protective liner. The pull up diaper portion and leg extensions each include a top protective liner and absorbent layer beneath the protective liner. The device 10b may be made of any synthetic disposable material(s). The external surface of the device may include a design imprinted thereon. The design may include any suitable images such, as stars, moons, characters, logos, etc.

To secure the donning of the device 10b by the wearer, a plurality of resealable fasteners 25 are provided on the pull up diaper portion 21 and the pair of leg extensions 23. The fasteners 25a provided on the pull up diaper portion 21 provide a secure attachment of the device 10b to the wearer at the wearer's waist. A fastener 25a is located at each side hip region 27 of the pull up diaper portion 21 and each fastener creates a secure closure at each side hip region. The fasteners 25b provided on the pair of leg extensions 23 each provide a secure attachment of the device 10b to the wearer at each of the wearer's thigh. A fastener 25b is located on each leg extension 23 along the side thigh region 29 and each fastener creates a secure closure at each side thigh region 29 of the respective thigh.

The attachment of the pull up diaper portion 21 and the pair of leg extensions 23 to the wearer should be snug so as to prevent any possible leakage. Any suitable resealable fasteners 25 may be used, such as, for example, fastener tape, Velcro strips, etc. The pull up diaper portion 21 may include an elastic band 31a along the waist of the pull up diaper portion and each leg extension 23 may include an elastic band 31b along the bottom of each leg extensions 23. The elastic bands 31 reinforce the attachment of the device 10b to the wearer and further prevents any leakage from the device 10b.

In another exemplary embodiment, device 10b may include a plurality of vertical elastic strips, where the pull up diaper portion 21 and each leg extension 23, has one or more elastic strips integrated therein, such that when the device is in a closed position, one pair of strips are positioned between the elastic band 31a on the right side of the waist area of the pull up diaper portion 21 and the elastic band 31b in the right leg extension 23 and another pair of strips are positioned between the elastic band 31a on the left side of the waist area of the pull up diaper portion 21 and the elastic band 31b in the left leg extension. The elastic strips provide for size adjustment and further waste containment reinforcement to prevent leakage.

The disclosed embodiments are not inclusive and many other modifications and variations will be apparent to someone of ordinary skill in the art with construction skills in the related arts. Together the descriptions and accompanying illustrations seek to provide an explanation of the basic principles of the embodiment and its application. It is therefore intended that the specification and embodiments be considered as exemplary only.

Those skilled in the art will appreciate from the foregoing description that the broad techniques of the embodiments of the present invention may be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

1. A leak-proof waste containment device comprising:
a diaper portion;
a waist extension;
a pair of leg extensions;
a plurality of elastic bands; and
a top protective liner and an absorbent layer beneath the top protective liner, wherein the top protective liner and the absorbent liner is integrated throughout the diaper portion, the waist extension and the pair of leg extensions;
wherein the diaper portion, the waist extension, and the pair of leg extensions collectively form an undergarment.

2. The device of claim 1, wherein the waist extension includes a pair of fasteners.

3. The device of claim 1, wherein the pair of leg extensions each include a pair of fasteners.

4. The device of claim 2, wherein the pair of fasteners are configured to secure the device to a wearer's waist.

5. The device of claim 3, wherein the pair of fasteners are configured to secure the device to each of a wearer's leg.

6. The device of claim 1, wherein the waist extension includes an elastic band.

7. The device of claim 1, wherein the pair of leg extensions each include an elastic band.

8. The device of claim 2, wherein the waist extension elastic band is configured to reinforce securement of the device to a wearer's waist to prevent leakage therefrom.

9. The device of claim 3, wherein the pair of leg extension elastic bands are configured to reinforce securement of the device to each of a wearer's leg to prevent leakage therefrom.

10. The device of claim 1, wherein each leg extension extends down a wearer's thigh.

11. A leak-proof waste containment device comprising:
a pull-up diaper portion;
a pair of leg extensions;
a plurality of fasteners;
a plurality of elastic bands; and
a top protective liner and an absorbent layer beneath the top protective liner, wherein the top protective liner and the absorbent liner is integrated throughout the diaper portion and the pair of leg extensions;
wherein the pull-up diaper portion and the pair of leg extensions collectively form an undergarment.
